(19) United States
(12) Patent Application Publication
(10) Pub. No.: US 2018/0325807 A1
(43) Pub. Date: Nov. 15, 2018

(54) METHOD AND APPARATUS FOR APPLYING A PHARMACEUTICAL AGENT TO REDUCE POST-SURGICAL ADHESIONS

(71) Applicant: PERGAMUM AB, Solna (SE)

(72) Inventors: Margit Mahlapuu, Gothenburg (SE); Monica Wiig, Uppsala (SE)

(21) Appl. No.: 15/977,809

(22) Filed: May 11, 2018

Related U.S. Application Data
(60) Provisional application No. 62/505,113, filed on May 11, 2017.

(51) Int. Cl. A61K 9/00; A61K 31/728; A61M 5/31

(52) U.S. Cl. CPC A61K 9/0019; A61K 31/728; A61K 38/10; A61M 2005/3114; A61M 5/31

(57) ABSTRACT

A pre-filled dispenser for applying a pharmaceutical agent to reduce the risk of post-surgical adhesions includes a first dispensing container having a first orifice and a second dispensing container having a second orifice fluidly coupled to the first orifice. A homogenous mixture is held in at least one of the first dispensing container and the second dispensing container. The homogenous mixture includes the peptide PXL01, or a pharmaceutically acceptable salt thereof, and a viscosity-enhancing pharmaceutically acceptable diluent or carrier.

This application claims priority to U.S. Provisional Application Ser. No. 62/505,113, filed on May 11, 2017, which is incorporated in its entirety herein by reference.

Embodiments of the present invention relate to methods and apparatuses for applying one or more pharmaceutical agents and/or pharmaceutical formulations to tissue, such as application to reduce post-surgical adhesions.

There are many situations where injured tissues exhibit a propensity to fuse to each other. For example, when a severe burn occurs in the mouth of a subject, there is a significant risk that a surface of the tongue may fuse, or conjoin, with a surface of the inner cheek. This causes a recurring injury as the two tissue masses, for example the cheek and the tongue, begin to fuse to each other and then they are torn asunder again. The cycle of injury continues because the moist and tender surfaces are prone to new cell growth and undesirable fusion.

Embodiments of the present invention provide methods and apparatuses for reducing undesired adhesions between tissues, such as post-surgical adhesions.

In some embodiments disclosed herein, a pre-filled dispenser for applying a pharmaceutical agent to reduce post-surgical adhesions includes a first dispensing container having a first orifice and a second dispensing container having a second orifice fluidly coupled to the first orifice. A mixture held in at least one of the first dispensing container and the second dispensing container comprises a quantity of a pharmaceutical composition effective to reduce post-surgical adhesions that is uniformly intermixed with a pharmaceutically acceptable diluent or carrier.

In some embodiments, a pre-filled dispenser is provided that includes a first dispensing container having a first orifice and a second dispensing container having a second orifice fluidly coupled to the first orifice. A quantity of a pharmaceutical composition effective to reduce post-surgical adhesions is held in the first dispensing container and a viscosity-enhancing pharmaceutically acceptable diluent or carrier is held in the second dispensing container such that the pharmaceutical composition and the pharmaceutically acceptable diluent or carrier are separated from one another.

In one aspect, the pharmaceutically acceptable diluent or carrier is hyaluronic acid having an average molecular weight of at least 300 kDa, such as at least 800 kDa. In some embodiments, the pharmaceutically acceptable diluent or carrier has a viscosity of at least 1.8 m³/kg, such as between 1.9 and 4.0 m³/kg.

In some embodiments disclosed herein, a method for applying a pharmaceutical agent to reduce post-surgical adhesions is provided.

In some embodiments, the method includes intermixing a quantity of a pharmaceutical composition, such as the peptide PXL01 or a pharmaceutically acceptable salt thereof, in a first dispensing container with a quantity of a pharmaceutically acceptable diluent or carrier in a second dispensing container that is fluidly coupled with the first dispensing container to produce a homogeneous mixture. The intermixing includes: (a) forcing at least some of the contents of the first dispensing container into the second dispensing container; (b) forcing at least some of the contents of the second dispensing container into the first dispensing container; and (c) repeating at least one of (a) or (b) until the mixture is a homogenous mixture. The method also includes coupling a first end of a catheter to at least one of the first dispensing container or the second dispensing container. The method includes applying the mixture to a surgical area, such as a wound area, through a second end of the catheter.

In some embodiments, the method includes intermixing a quantity of the peptide PXL01 in a first dispensing container with a quantity of a gelling agent in a second dispensing container that is fluidly coupled with the first dispensing container to produce a mixture. The intermixing includes: (a) forcing at least some of the contents of the first dispensing container into the second dispensing container; (b) forcing at least some of the contents of the second dispensing container into the first dispensing container; and (c) repeating at least one of (a) or (b) until the mixture has a predefined uniformity. The method also includes coupling a first end of a catheter to at least one of the first dispensing container or the second dispensing container. The method includes applying the mixture to a surgical area such as a wound area through a second end of the catheter.

### BRIEF DESCRIPTION OF THE DRAWINGS

Several alternative embodiments will hereinafter be described in conjunction with the appended drawings and figures, wherein like numerals denote like elements, and in which:

FIG. 1 is a front view of an exemplary embodiment of a pre-filled dispenser in accordance with some embodiments;

FIG. 2 is a perspective view of another exemplary embodiment of a pre-filled dispenser in accordance with some embodiments;

FIG. 3 schematically depicts a mixture applied to an artificial joint to reduce the risk of tissue adhesion in accordance with some embodiments;

FIG. 4 schematically depicts a mixture being applied to a first tissue mass to reduce the risk of the first tissue mass adhering to a second tissue mass in accordance with some embodiments; and

FIG. 5 is a flow chart illustrating an exemplary embodiment of a method for applying a pharmaceutical agent to reduce post-surgical adhesions.

### DETAILED DESCRIPTION

In some embodiments described herein, a method and apparatus for reducing the risk of tissue adhesion is provided. In some embodiments, a homogeneous mixture comprising the peptide PXL01, or a pharmaceutically acceptable salt thereof, and a viscosity-enhancing pharmaceutically acceptable diluent or carrier is provided for application to reduce the risk of tissue adhesion.

The risk of fusion or conjoining of tissue masses that are proximate to each other, but are not normally contiguous, may be reduced by the application of a substance known as PXL01. PXL01 is an anti-adhesion/-scarring therapeutic peptide having a chemical structure of [N-Acetyl-L-glutamyl-L-alanyl-L-threonyl-L-lysyl-L-cysteinyl-L-phenylalanyl-L-glutaminyl-L-trypto-phanyl-L-gIutaminyl-L-arginyl-L-asparaginyl-L-methionyl-L-arginyl-L-lysyl-L-valyl-L-arginyl-glycyl-L-prolyl-L-prolyl-L-valyl-L-seryl-L-cysteinyl-L-isoleucyl-L-lysyl-L-argininamide (5->22) disulfide, acetate salt]. Exemplary pharmaceutical compositions of PXL01 are described in U.S. Pat. No. 9,878,019 of Pergamum AB, which is incorporated in its entirety herein by reference.

PXL01 peptide compositions tend to exhibit very low viscosity and are watery due to the PXL01, or a pharmaceutically acceptable salt thereof, being dissolved in saline and/or another low viscosity fluid buffer, such as acetate buffer. The application of a low viscosity pharmaceutical agent onto a surface is likely ineffective because a low viscosity pharmaceutical agent has a tendency to “run” from its application site. This running reduces the amount of PXL01 at the application site, where it is needed, which may increase the amount of PXL01 that must be applied to be effective.

In order to enhance the application process, it is useful to increase the viscosity and bioadhesivitiy of the PXL01, or pharmaceutically acceptable salt thereof, so that it is more likely to remain on the surface upon which it is applied. To increase the viscosity and bioadhesivity of the PXL01, the PXL01 is intermixed with a viscosity-enhancing pharmaceutically acceptable diluent or carrier to create a mixture.

In some exemplary embodiments, PXL01 is combined with a viscosity-enhancing pharmaceutically acceptable diluent or carrier, which may be hyaluronic acid having an average molecular weight of at least 300 kDa, such as at least 800 kDa. The hyaluronic acid, having a relatively high viscosity compared to the PXL01, mixes with the PXL01 to form a mixture with a higher viscosity than PXL01 by itself. In some embodiments, a combination of several pharmaceutically acceptable diluents or carriers may be useful.

One class of pharmaceutically acceptable diluents or carriers that exhibits useful viscosity properties is that of viscous water-soluble polymers of synthetic origin. These include, but are not limited to, polyvinylpyrrolidone (PVP), polyvinyl alcohol (PVA), and polylactic acid (PLA).

Another class of pharmaceutically acceptable diluents or carriers that exhibits useful viscosity properties is that of viscous water-soluble polymers of natural origin. These include, but are not limited to hyaluronic acid (HA), alginate, and cellulose-derivatives, such as carboxymethyl cellulose (CMC). In some embodiments, the diluent or carrier is sodium hyaluronate, which is a sodium salt of hyaluronic acid that is pharmaceutically acceptable.

In some embodiments, the pharmaceutically acceptable diluent or carrier is a gelling agent. It should be appreciated that, as used herein, a “gelling agent” is a substance of relatively high viscosity, as compared to the peptide PXL01, that can intermix with the peptide PXL01 to create a uniform mixture with a higher viscosity than the peptide PXL01 by itself. In some embodiments, the gelling agent is a water-soluble polymer. The previously described viscous water-soluble polymers of synthetic and natural origin are considered “gelling agents,” in the context of this disclosure, even though the resultant mixture between the peptide PXL01 and the substance may not necessarily form what would normally be considered a gel.

Although the viscosity-increasing characteristics of the above diluents or carriers are excellent when combined with PXL01, one other important characteristic is that these exemplary diluents or carriers do not affect, at least radically, the stability of PXL01. Many other viscosity-enhancing diluents or carriers can degrade the peptide molecule PXL01.

Even though these exemplary pharmaceutically acceptable diluents or carriers provide for relative stability of the PXL01 peptide, it is still not entirely ideal to keep the PXL01 peptide mixed with the diluent or carrier for a prolonged time period. In order to increase the efficacy of application of PXL01, as mixed with a selected pharmaceutically acceptable diluent or carrier, it is best to mix the PXL01 pharmaceutical agent with the pharmaceutically acceptable diluent or carrier just prior to application. Mixing the PXL01 and pharmaceutically acceptable diluent or carrier just prior to application may reduce the effects of, for example, phase or density separation, as the pharmaceutically acceptable diluent(s) or carrier(s) will tend to be relatively dense compared to the PXL01 composition.

Some embodiments described herein provide devices for mixing and dispensing PXL01 with a pharmaceutically acceptable diluent or carrier to reduce the risk of tissue adhesion.

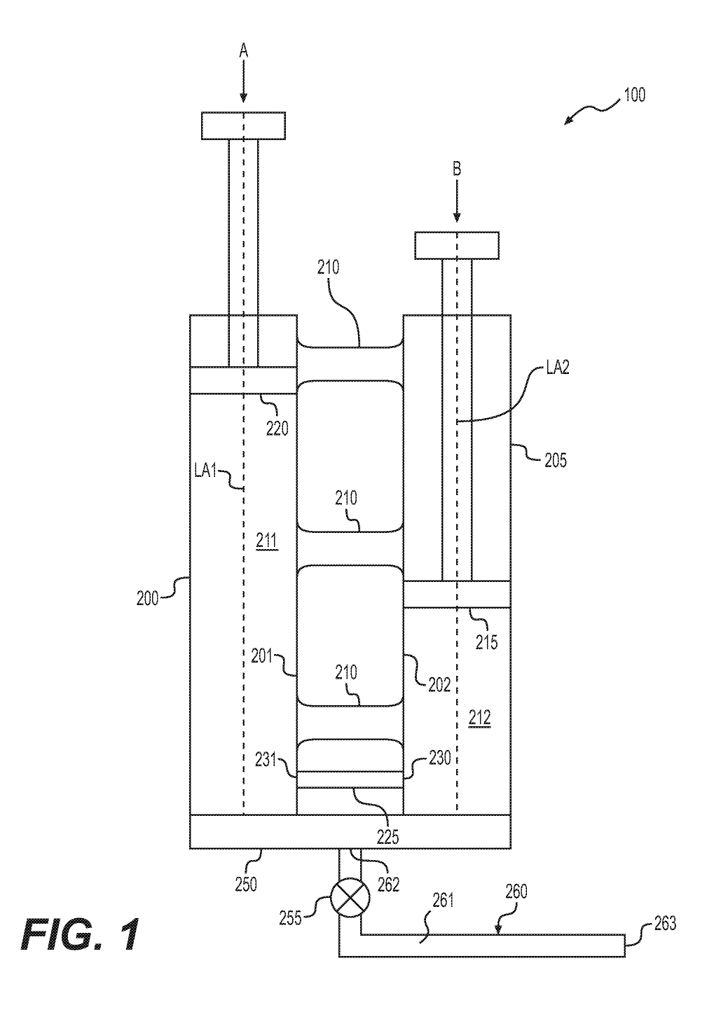

FIG. 1 depicts an exemplary embodiment of a dispenser 100 that includes a first dispensing container 200 fluidly coupled to a second dispensing container 205. In some embodiments, the first dispensing container 200 and the second dispensing container 205 may each be in the form of or include a syringe, with the two syringes 200, 205 each defining a respective longitudinal axis LA1, LA2 and being set in parallel offset, i.e., non-coaxial, with respect to each other. Each syringe 200, 205 may include a respective plunger 220, 215 that is disposed in a respective internal volume 211, 212 of the syringe 200, 205. Each of the syringes 200, 205 has a respective orifice 231, 230 formed therein that is fluidly coupled to a corresponding orifice of the other syringe. In some embodiments, the dispenser 100 further includes a channel 225 fluidly coupling the orifices 231, 230 together so the internal volumes 211, 212 of the syringes 200, 205 are fluidly coupled with one another, the significance of which is described further herein. In some embodiments, the orifice 231 is formed in a side 201 of syringe 200, which may be referred to as the “first syringe,” and the orifice 230 is formed in a side 202 of syringe 205, which may be referred to as the “second syringe.” The channel 225 fluidly coupling the orifices 231, 230 may extend transversely, such as perpendicularly, to the longitudinal axes LA1, LA2 of the syringes 200, 205.

In some embodiments, one or more binding webs 210 connect the syringes 200, 205 together and maintain a fixed physical relationship of the two syringes 200, 205 relative to each other. While three webs 210 are shown connecting the syringes 200, 205, the number of webs 210 may be altered, as needed, and may include one web, two webs, four webs, etc.

The dispenser 100 may be pre-filled with a quantity of a pharmaceutical composition, such as the peptide PXL01, effective to reduce the risk of post-surgical adhesions that is separated from a quantity of a pharmaceutically acceptable diluent or carrier. In some embodiments, the PXL01 is initially provided in the first syringe 200 and the pharmaceutically acceptable diluent or carrier is initially provided in the second syringe 205. In some embodiments, the PXL01 and the pharmaceutically acceptable diluent or carrier may both be provided in one of the syringes 200, 205, but separated due to, for example, phase or density separation. In some embodiments, the quantity of PXL01 is between 0.1 mL and 50 mL and the quantity of diluent or carrier is between 0.15 mL and 75 mL, depending on the application site. In some embodiments, the volume of the pharmaceutical composition to the volume of pharmaceutically acceptable diluent or carrier in the dispenser 100 is around 1:1.5. In some embodiments, the syringes 200, 205 have internal volumes 211, 212 that are the same. In other embodiments, the internal volumes 211, 212 of syringes 200, 205 are different. The internal volumes 211, 212 may be chosen to both be greater than the sum of the volumes of the pharmaceutical composition and the pharmaceutically acceptable diluent or carrier.

As previously described, PXL01 has a relatively low viscosity while the pharmaceutically acceptable diluent or carrier has a relatively high viscosity, compared to the PXL01. To produce a mixture with a higher viscosity than PXL01 alone, the PXL01 and the pharmaceutically acceptable diluent or carrier are intermixed with one another to form a substantially homogeneous mixture. In some embodiments, the pharmaceutically acceptable diluent or carrier is hyaluronic acid having a molecular weight of at least 300 kDa, such as at least 800 kDa, and having a viscosity between 1.9 and 4.0 m³/kg In some embodiments, the homogeneous mixture comprises a gel, a salve, a balm, a cream, an ointment, a paste, or any other type of composition with a suitable viscosity and consistency for applying to a tissue site.

In some embodiments, the hyaluronic acid is present at a concentration of between 0.5 and 2.5% (w/w) of the homogeneous mixture and the peptide PXL01 is present at a concentration of between 0.5 mg/mL to 25 mg/mL of the homogeneous mixture.

To intermix the PXL01 and the pharmaceutically acceptable diluent or carrier, the plunger 220 of the first syringe 200 may be depressed in a direction A to force some or all of the contents of the first syringe 200, in this case the PXL01 composition, through the orifice 231 and the channel 225 into the second syringe 205 through the orifice 230. While the PXL01 and the pharmaceutically acceptable diluent or carrier are in the second syringe 205, the plunger 215 of the second syringe 205 may be depressed in a direction B to force some or all of the contents of the second syringe 205 through the orifice 231 and the channel 225 into the first syringe 200 through the orifice 231. This process of depressing the plungers 220, 215 to exchange the contents of the syringes 200, 205 with one another can be repeated until a homogeneous mixture is formed that has the quantity of pharmaceutical composition, such as PXL01, uniformly intermixed with the pharmaceutically acceptable diluent or carrier, which may be hyaluronic acid having a molecular weight of at least 300 kDa, such as at least 800 kDa, and having a viscosity between 1.9 and 4.0 m³/kg.

In some embodiments, the orifice 231 of the first syringe 200, which may be referred to as a “first orifice,” defines a first opening area and the orifice 230 of the second syringe 205, which may be referred to as a “second orifice,” defines a second opening area. In some embodiments, the second opening area of the second orifice 230 is substantially equal to the first opening area of the first orifice 231. Having the respective opening areas of the orifices 230, 231 equal to one another may allow for a relatively low fluid pressure to be exerted on the contents of the syringes 200, 205 during the intermixing. When the diluent or carrier is, for example, hyaluronic acid, the relatively low fluid pressure reduces the risk that the structure of the hyaluronic acid is compromised, which may lead to a reduction in the viscosity of the final mixture. In some embodiments, an orifice diameter of the first orifice 231 and the second orifice 230 may be between 0.5 mm and 10 mm.

In some embodiments, the dispenser 100 further includes a catheter coupling 250 for coupling the syringes 200, 205 to a catheter 260 to deliver the mixture through a fluid channel 261 defined in the catheter 260. In some embodiments, a first end 262 of the catheter 260 is fluidly coupled to the catheter coupling 250 and a second end 263 of the catheter 260 opposite the first end 262 is open to deliver the mixture. In some embodiments, the catheter 260 defines a delivery opening diameter at the second end 263 that may be, for example, 2 mm. In some embodiments, a valve 255 may be disposed in the fluid channel 261 to switch the dispenser 100 between a mixing configuration and a dispensing configuration. In the mixing configuration, the valve 255 is closed to block the contents of the syringes 200, 205 from flowing into the fluid channel 261 as the plungers 220, 215 are depressed. When the mixture is ready for dispensing, the valve 255 is opened so the contents of the syringes 200, 205 can be forced into the fluid channel 261 and out of the second end 263 of the catheter 260. Thus, it should be appreciated that the catheter coupling 250 may be fluidly coupled to both syringes 200, 205 prior to or during intermixing of the PXL01 and the pharmaceutically acceptable diluent or carrier or, in some embodiments, may be coupled to the syringes 200, 205 following intermixing.

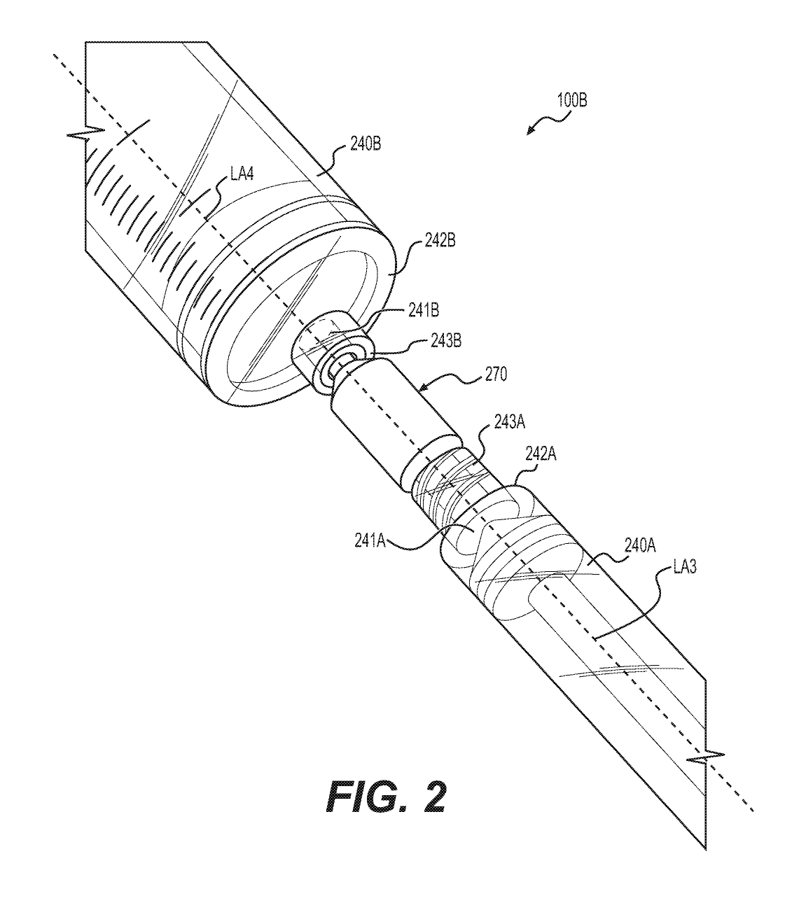

Referring now to FIG. 2, another exemplary embodiment of a dispenser 100B for applying pharmaceutical agent to reduce the risk of post-surgical adhesions is illustrated that includes a first syringe 240A fluidly coupled to a second syringe 240B by a transfer connector 270. The first syringe 240A includes a quantity of a pharmaceutical composition, such as the peptide PXL01, effective to reduce the risk of post-surgical adhesions and the second syringe 240B includes a pharmaceutically acceptable diluent or carrier, such as hyaluronic acid, to intermix with and increase the viscosity of the pharmaceutical composition, as previously described. As illustrated, the first syringe 240A and the second syringe 240B have different internal volumes, but in some other embodiments the two syringes 240A, 240B have equal internal volumes.

The first syringe 240A has a first orifice 241A formed in its longitudinal end 242A and the second syringe 240B has a second orifice 241B formed in its longitudinal end 242B, with the orifices 241A, 241B coupled to one another by the syringe coupler 250. The first orifice 241A may have a first Luer lock 243A associated therewith and the second orifice 241B may have a second Luer lock 243B associated therewith, with the transfer connector 270 being a Luer coupler configured to fluidly couple the respective Luer locks 243A, 243B and orifices 241A, 241B together. This coupling enables the contents of each syringe 240A, 240B to be repeatedly transferred from one syringe to the other as needed to intermix the contents of the syringes 240A, 240B to a predefined uniformity, similar to the previously described dispenser 100. Each of the syringes 240A, 240B may define a respective longitudinal axis LA3, LA4, with the longitudinal axes LA3, LA4 being coaxial with one another.

After sufficient mixing of the pharmaceutical composition and the pharmaceutically acceptable diluent or carrier to form a homogeneous mixture, the homogeneous mixture may be contained in one or both of the syringes 240A, 240B. In some embodiments, the homogeneous mixture is contained in both syringes 240A, 240B, which are decoupled from one another and used separately to apply the mixture by way of a catheter, as previously described. Alternatively, in some embodiments the contents of one of the syringes 240A, 240B may be emptied into the other syringe 240B, 240A so the mixture may be applied from one of the syringes 240A, 240B coupled to a catheter after mixing.

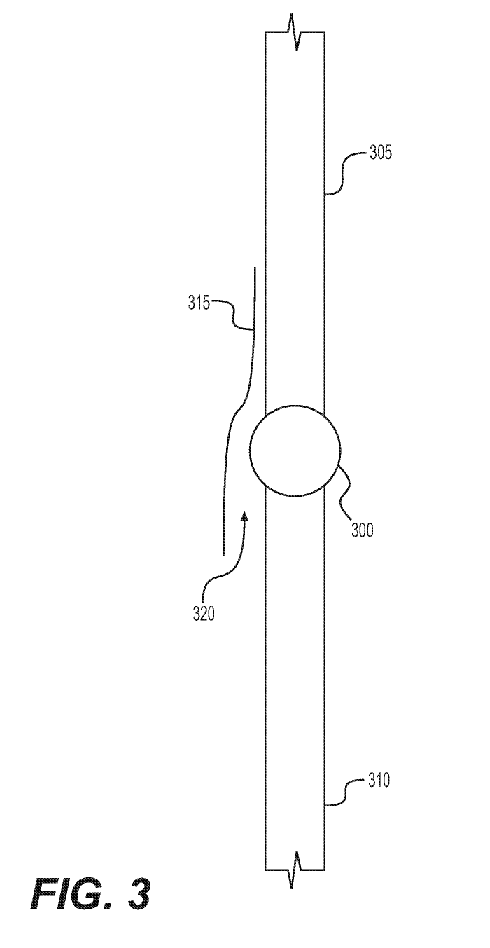

FIG. 3 schematically depicts an exemplary manner in which the mixture of the pharmaceutical composition and the pharmaceutically acceptable diluent or carrier is applied to reduce the risk of post-surgical adhesion. In this exemplary illustration, an artificial joint 300 is used to join two limbs, such as an inner limb 305 to an outer limb 310. It is undesired for tissue 315 attached to either the inner limb 305 or the outer limb 310 to adhere to the artificial joint 300. The homogeneous mixture of the pharmaceutical composition, which may be the peptide PXL01, and the pharmaceutically acceptable diluent or carrier, which may be hyaluronic acid, as disclosed herein, is applied to an application region 320 where there is a potential for adhesion between the tissue 315 and the artificial joint 300. In this sense, application of the PXL01 and hyaluronic acid mixture to the application region 320 reduces the risk of the tissue 315 undesirably attaching to the artificial joint 300. The mixture may be applied to the application region 320 by a catheter, as previously described. In some embodiments, the application region 320 is one or more of: an injured tendon or nerve repaired by surgery; a region between an artificial knee joint and tissue interfacing with the artificial knee joint; a region between an artificial hip joint and tissue interfacing with the artificial hip joint; a region between an artificial shoulder joint and tissue interfacing with the artificial shoulder joint; an artificial temporomandibular joint (TMJ) and tissue interfacing with the artificial TMJ; and a region between a dura of a spinal cord and an interface with a vertebra. While the mixture is illustrated being applied to an application region 320 between an artificial joint and tissue 315, it should be appreciated that the mixture may be applied to a first surface of a first tissue mass that is proximate to a second surface of a second tissue mass that is not naturally conjoined to the first surface of the first tissue mass to reduce the risk of the first surface and the second surface conjoining.

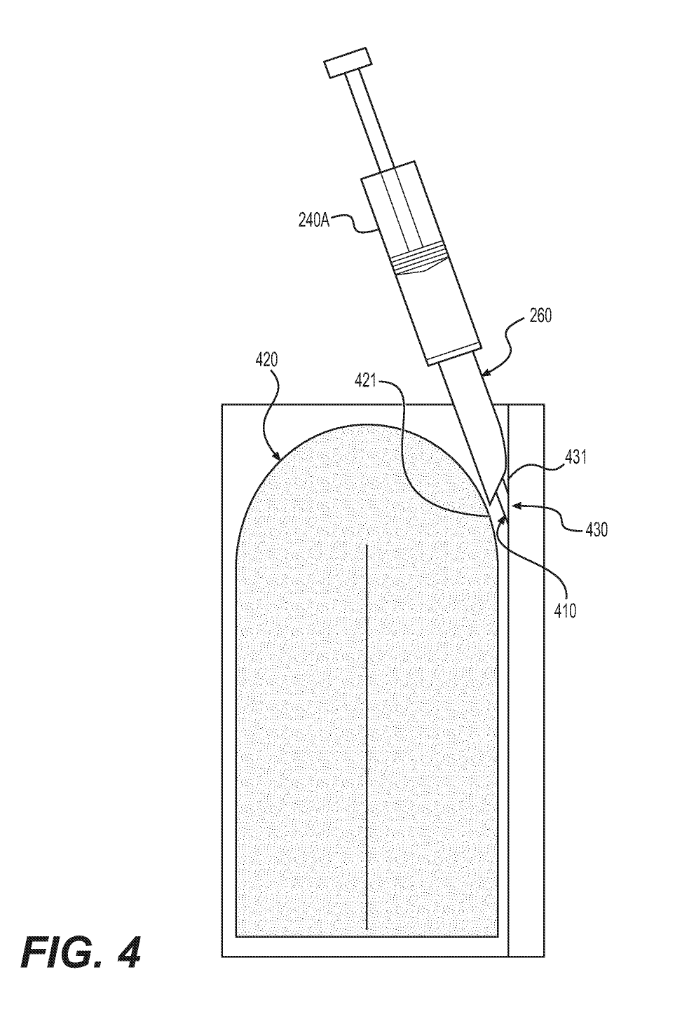

In some embodiments, and referring now to FIG. 4, the mixture is applied to an application region 410 between a first tissue mass 420 and a second tissue mass 430 that is proximate to the first tissue mass 420. The mixture may be applied to a surface 421 of the first tissue mass 420 that is proximate to a surface 431 of the second tissue mass 430, but not naturally conjoined to the surface 421. For example, the mixture may be applied to a surface of the inner cheek of a subject to reduce the risk of adhesion between the surface of the inner cheek and a surface on the tongue of the subject. It should be appreciated that the inner cheek and tongue are exemplary tissue masses only, and the mixture can be readily applied to other areas to reduce the risk of post-surgical adhesion.

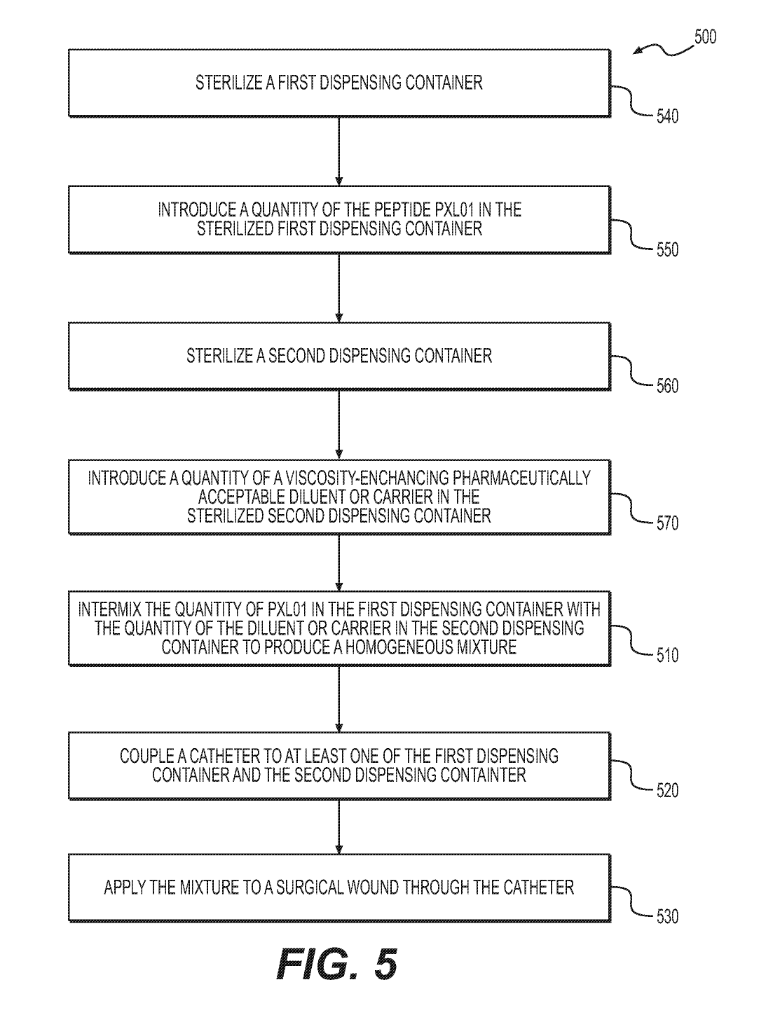

Referring now to FIG. 5, an exemplary embodiment of a method 500 for applying a pharmaceutical agent to reduce the risk of post-surgical adhesions is provided. The method 500 includes intermixing 510 a quantity of a pharmaceutical composition in a first dispensing container, such as a syringe 200, 240A, with a quantity of a pharmaceutically acceptable diluent or carrier in a second dispensing container, such as a syringe 205, 240B, that is fluidly coupled with the first dispensing container 200, 240A. As previously described, the pharmaceutical agent may be the peptide PXL01, or a pharmaceutically acceptable salt thereof, and the pharmaceutically acceptable diluent or carrier may be hyaluronic acid having a molecular weight of at least 300 kDa, such as at least 800 kDa, and having a viscosity between 1.9 and 4.0 m³/kg. In some embodiments, the pharmaceutically acceptable diluent or carrier comprises a gelling agent. The intermixing 510 produces a homogeneous mixture. In some embodiments, the intermixing 510 comprises: (a) forcing at least some of the contents of the first dispensing container 200, 240A into the second dispensing container 205, 240B; forcing at least some of the contents of the second dispensing container 205, 240B into the first dispensing container 200, 240A; and (c) repeating at least one of (a) or (b) until the mixture is substantially homogeneous. A first end 262 of a catheter 260 is coupled 520 to at least one of the first dispensing container 200, 240A and the second dispensing container 205, 240B. In some embodiments, the catheter 260 is coupled 520 to at least one of the first dispensing container 200, 240A and the second dispensing container 205, 240B prior to or during the intermixing 510. In some embodiments, the first end 262 of the catheter 260 couples 520 to one or both of the dispensing containers 200, 205 via a catheter coupling 250, as previously described. In some embodiments, a first end of a catheter couples to a Luer lock 243A, 243B of a syringe 240A, 240B. The mixture is applied 530 to a surgical wound through a second end 263 of the catheter 260.

It should be appreciated that the transfer of the contents from one dispensing container 200, 205, 240A, 240B to the other effects intermixing of the two original and initially separate compounds. Even in instances where the quantities of the pharmaceutical composition and the pharmaceutically acceptable diluent or carrier are initially provided in the same container, repeatedly transferring the contents of one dispensing container 200, 205, 240A, 240B into the other effects intermixing of the pharmaceutical composition and the pharmaceutically acceptable diluent or carrier to overcome separation of the two compositions due to, for example, phase or density separation.

In some embodiments, the pharmaceutical composition is provided in the second dispensing container 205, 240B and the pharmaceutically acceptable diluent or carrier is provided in the first dispensing container 200, 240A. It should be appreciated that, because the contents of the containers 200, 205, 240A, 240B become intermixed together, the pharmaceutical composition and the pharmaceutically acceptable diluent or carrier may initially begin in either container 200, 205, 240A, 240B without a significant effect on the intermixing.

In some exemplary embodiments, the contents of one dispensing container 200, 205, 240A, 240B are forced into the other dispensing container 205, 200, 240B, 240A by applying pressure to the internal volume in the first dispensing container 200, 240A, irrespective of whether the pharmaceutical composition or the pharmaceutically acceptable diluent or carrier is initially provided in the first dispensing container 200, 240A. This application of pressure forces the contents of the first dispensing container 200, 240A into the second dispensing container 205, 240B. The transfer of the contents may be supported via a closed perimeter channel 225 fluidly coupling respective orifices 231, 230 of the first dispensing container 200 and the second dispensing container 205. In some embodiments, the transfer of the contents is supported by a transfer connector 270 that fluidly couples respective Luer locks 243A, 243B of the dispensing containers 240A, 240B.

Once some or all of the contents of the first dispensing container 200, 240A are transferred to the second dispensing container 205, 240B, pressure applied to the contents of the first dispensing container 200, 240A is relaxed and pressure is applied to the contents of the second dispensing container 205, 240B. Accordingly, the contents of the second dispensing container 205, 240B, which includes both the pharmaceutical composition and the pharmaceutically acceptable diluent or carrier, are forced back into the first dispensing container 200, 240A. This back-and-forth process continues until a suitable uniformity in the resulting mixture is obtained. In some embodiments, a second pressure container acting on the contents of the second dispensing container 205, 240B is relieved as pressure is applied to the contents of the first dispensing container 200, 240A and a first pressure container acting on the contents of the first dispensing container 200, 240A is relieved as pressure is applied to the contents of the second dispensing container 205, 240B.

The area of the closed-perimeter channel 225 and between the orifices 231, 230 of the two dispensing containers 200, 205 should be carefully selected according to the viscosity of the selected pharmaceutically acceptable diluent(s) or carrier(s).

In some exemplary embodiments, where the selected pharmaceutically acceptable diluent or carrier comprises hyaluronic acid, the orifice 231, 230, 241A, 241B provided at one end of each dispensing container 200, 205, 240A, 240B is selected to ensure that suitable mixing occurs without an unreasonable repetition of the back-and-forth intermixing. Exemplary diameters that may be used for a substantially circular orifice 231, 230, 241A, 240B are in the range of 0.5 mm to 10 mm. This range is also well suited to make sure that the amount of pressure needed to effect intermixing can be provided by a person having normal strength without a significant risk of so much pressure being applied that the dispensing containers 200, 205, 240A, 240B burst during mixing. Further, such areas of the orifices 231, 230, 241A, 241B can allow for suitable intermixing of PXL01 and hyaluronic acid without applying so much pressure that the physical properties, such as viscosity, of the hyaluronic acid are significantly altered.

In some exemplary embodiments, the method 500 further includes sterilizing 540 an internal surface and an exposed surface of one or more of the dispensing containers 200, 205, 240A, 240B, such as the first dispensing container 200, 240A. Once the first dispensing container 200, 240A is sterilized 540, the quantity of pharmaceutical composition can be introduced 550 into the first dispensing container 200, 240A. In some exemplary embodiments, an internal surface and an exposed surface of the second dispensing container 205, 240B are sterilized 560. Once the second dispensing container 205, 240B is sterilized, the pharmaceutically acceptable diluent or carrier can be introduced 570 into the sterile second dispensing container 205, 240B.

While the present method and apparatus have been described in terms of several alternative and exemplary embodiments, it is contemplated that alternatives, modifications, permutations, and equivalents thereof will become apparent to those skilled in the art upon a reading of the specification and study of the drawings. It is therefore intended that the true spirit and scope of the claims appended hereto include all such alternatives, modifications, permutations, and equivalents.

What is claimed is:

1. A pre-filled dispenser for applying a pharmaceutical agent to reduce the risk of post-surgical adhesions, comprising:
a first dispensing container having a first orifice;
a second dispensing container having a second orifice fluidly coupled to the first orifice; and
a homogenous mixture held in at least one of the first dispensing container and the second dispensing container, the homogenous mixture comprising the peptide PXL01, or a pharmaceutically acceptable salt thereof, and a viscosity-enhancing pharmaceutically acceptable diluent or carrier.

2. The dispenser of claim 1, wherein the first orifice defines a first opening area and the second orifice defines a second opening area that is substantially equal to the first opening area.

3. The dispenser of claim 1, wherein the diluent or carrier comprises hyaluronic acid having an average molecular weight of at least 300 kDa.

4. The dispenser of claim 3, wherein the homogenous mixture comprises:
the peptide PXL01, or a pharmaceutically acceptable salt thereof; and
hyaluronic acid having an average molecular weight of at least 800 kDa, wherein the hyaluronic acid is present at a concentration of between 0.5 and 2.5% (w/w) of the homogeneous mixture and the peptide PXL01 is present at a concentration of between 0.5 mg/mL to 25 mg/mL of the homogeneous mixture.

5. The dispenser of claim 1, wherein the first dispensing container defines a first longitudinal axis and the second dispensing container defines a second longitudinal axis that is coaxial with the first longitudinal axis.

6. The dispenser of claim 5, wherein the first orifice is formed in a longitudinal end of the first dispensing container and the second orifice is formed in a longitudinal end of the second dispensing container.

7. The dispenser of claim 1, wherein the first dispensing container defines a first longitudinal axis and the second dispensing container defines a second longitudinal axis that is non-coaxially parallel with the first longitudinal axis.

8. The dispenser of claim 1, wherein the diluent or carrier comprises a gelling agent.

9. The dispenser of claim 8, wherein the gelling agent comprises hyaluronic acid.

10. A method for applying a pharmaceutical agent to reduce the risk of post-surgical adhesions, the method comprising:
intermixing a quantity of the peptide PXL01 in a first dispensing container with a viscosity-enhancing pharmaceutically acceptable diluent or carrier in a second dispensing container that is fluidly coupled with the first dispensing container to produce a homogeneous mixture, the intermixing comprising:
(a) forcing at least some of the contents of the first dispensing container into the second dispensing container;
(b) forcing at least some of the contents of the second dispensing container into the first dispensing container; and
(c) repeating at least one of (a) or (b) until the mixture is substantially homogenous;
coupling a first end of a catheter to at least one of the first dispensing container or the second dispensing container; and
applying the mixture to a surgical wound through a second end of the catheter.

11. The method of claim 10, further comprising at least one of:
relieving a second container pressure acting on the contents of the second dispensing container during (a); or
relieving a first container pressure acting on the contents of the first dispensing container during (b).

12. The method of claim 10, further comprising coupling a first orifice formed in the first dispensing container to a second orifice formed in the second dispensing container prior to the intermixing.

13. The method of claim 12, wherein the first orifice is formed in a longitudinal end of the first dispensing container and the second orifice is formed in a longitudinal end of the second dispensing container.

14. The method of claim 13, wherein the first orifice defines a first opening area and the second orifice defines a second opening area that is substantially equal to the first opening area.

15. The method of claim 10, wherein the viscosity-enhancing pharmaceutically acceptable diluent or carrier is selected from the group comprising hyaluronic acid, polyvinylpyrrolidone, polyvinyl alcohol, polylactic acid, alginate, and a cellulose-derivative.

16. The method of claim 10, wherein applying the mixture from the second end of the catheter to the surgical wound comprises applying the mixture to a surface of a first tissue mass, wherein the surface of the first tissue mass is proximate to a surface of a second tissue mass and the first surface and the second surface are not naturally conjoined.

17. The method of claim 10, wherein applying the mixture from the second end of the catheter to the surgical wound comprises applying the mixture to at least one of:
an injured tendon or nerve repaired by surgery;
an artificial knee joint and tissue interfacing therewith;
an artificial hip joint and tissue interfacing therewith;
an artificial shoulder joint and tissue interfacing therewith;
an artificial temporomandibular joint and tissue interfacing therewith; and
a dura of a spinal cord at an interface with a vertebrae.

18. A method for applying a pharmaceutical agent to reduce the risk of post-surgical adhesions, comprising:
intermixing a quantity of the peptide PXL01 in a first dispensing container with a quantity of a gelling agent in a second dispensing container that is fluidly coupled with the first dispensing container to produce a mixture, the intermixing comprising:
(a) forcing at least some of the contents of the first dispensing container into the second dispensing container;
(b) forcing at least some of the contents of the second dispensing container into the first dispensing container; and
(c) repeating at least one of (a) or (b) until the mixture has a predefined uniformity;
coupling a first end of a catheter to at least one of the first dispensing container or the second dispensing container; and
applying the mixture to a surgical wound through a second end of the catheter.

19. The method of claim 18, wherein the gelling agent comprises hyaluronic acid having a higher viscosity than the peptide PXL01.

20. The method of claim 19, wherein the first dispensing container is a first syringe and the second dispensing container is a second syringe.
